(19) United States
(12) Patent Application Publication
(10) Pub. No.: US 2005/0043612 A1
(43) Pub. Date: Feb. 24, 2005

(54) Multipurpose connection/reception device for nuclear magnetic resonance imager

(76) Inventors: Herve Saint-Jalmes, Lyon (FR); Olivier Beuf, Montanay (FR)

Correspondence Address: CLARK & BRODY, 1750 K STREET NW, SUITE 600, WASHINGTON, DC, 20006, US

(21) Appl. No.: 10/490,418

(22) PCT Filed: Sep. 25, 2002
(86) PCT No.: PCT/FR02/03264

(30) Foreign Application Priority Data
Sep. 26, 2001 (FR) 01/12361

(51) Int. Cl. A61B 5/05

(52) U.S. Cl. 600/422

(57) ABSTRACT

The universal connector/receiver device for a nuclear magnetic resonance imager (1) of the invention is provided with at least one radiofrequency transmitter coil (5) and with at least one standard radiofrequency receiver coil (6). According to the invention, the device comprises at least one independent radiofrequency receiver coil (14) adapted to the shape and/or size of an item to be investigated, for the purpose of transforming an electromagnetic signal induced by nuclear magnetic resonance into an electrical signal, said independent radiofrequency receiver coil (14) being connected to a receiver coupling loop (20) serving to provide magnetic coupling with the standard radiofrequency receiver coil (6).

The present invention relates to the technical field of nuclear magnetic resonance (NMR) imaging, and also to applications of this technique in the medical field.

More particularly, the invention relates to a universal connector/receiver device making it possible to adapt various types of radiofrequency (RF) receiver coil to a given imager.

In general, nuclear magnetic resonance imagers are associated with a magnetic system that generates a magnetic field that varies in three dimensions, in a space occupied by the item under investigation, so as to obtain a different resonant frequency at each point thereof. A radiofrequency transmission coil is immersed in the magnetic field in order to transmit an electromagnetic wave which, as a function of the frequency of said wave and of the magnetic field at each point of the item under investigation, induces resonance that is accompanied by the transmission of a magnetic signal. A radiofrequency receiver coil serves to detect this signal induced by resonance and to transform it into an electrical signal. The radiofrequency transmitter or receiver coils to which the invention relates comprise means for transforming electrical signals into an electromagnetic wave for a transmitter coil or, vice versa for a receiver coil. These means are also known as antennas.

The radiofrequency transmitter coil and the radiofrequency receiver coil are generally connected respectively to a radiofrequency transmission control system and to a device for building a two- or three-dimensional image of the item under investigation, these means together constituting a nuclear magnetic resonance imager.

In general, the radiofrequency receiver coil is adapted to the shape and/or size of the item under investigation. This item can be any item that is observable using a nuclear magnetic resonance imager. In general, it corresponds to a portion of the body of a patient.

Thus, the radiofrequency receiver coil is often selected from amongst a set of “standard” radiofrequency receiver coils of characteristics and performance that vary as a function of the shape and/or size of the item under investigation.

Each model of imager is often provided with a proprietary connector that can be fitted to connectors coming from the same set of standard radiofrequency receiver coils.

A user possessing one particular model of imager generally cannot fit radiofrequency receiver coils that do not correspond to that model or to the design date of the imager in question. In addition, a set of standard radiofrequency receiver coils corresponding to one particular model of imager generally does not make it possible to cover the entire extent of medical applications of interest to all of the users in a given hospital establishment.

The problem faced by the user is thus associated with lack of compatibility or with the magnitude of the investment required to acquire all of the imagers needed for the intended applications.

It is specifically the object of the invention to propose a device enabling this lack of compatibility to be mitigated.

To satisfy the needs of users of imaging techniques, the invention provides a universal connector/receiver device for a nuclear magnetic resonance imager provided with at least one radiofrequency transmitter coil and with at least one standard radiofrequency receiver coil,
the device being characterized in that it comprises at least one independent radiofrequency receiver coil adapted to the shape and/or size of an item to be investigated, for the purpose of transforming an electromagnetic signal induced by nuclear magnetic resonance into an electrical signal, said independent radiofrequency receiver coil being connected to a receiver coupling loop serving to provide magnetic coupling with the standard radiofrequency receiver coil.

Although the universal connector/receiver device described above enables the field of user applications to be extended, the frequency of the electromagnetic waves used generally constitutes a characteristic that is invariable and predetermined for any particular type of imager used. Thus, the development of nuclear magnetic resonance imagers is tending towards applying stronger magnetic fields, which leads to the need to change the frequency of the electromagnetic waves used. Furthermore, independently of the magnetic field used, the user can be led to envisage working at different frequencies as a function of the type of nucleus that is to be made to resonate. As a result, imagers that are generally suitable for working at a single frequency only, once more constitute a limitation on the fields of application that can be envisaged.

It is specifically an object of a particular embodiment of the invention to use a universal connector/receiver device that makes it possible to transmit an electromagnetic wave at a frequency that can be adapted to the intended application.

This particular embodiment thus provides a universal connector/receiver device as described above, characterized in that it includes at least one independent radiofrequency transmitter coil connected to at least one transmitter coupling loop providing magnetic coupling with the radiofrequency transmitter coil, and in that a frequency synthesizer is interposed between the output of the transmitter coupling loop and an input of the receiver coupling loop in order to transmit an electromagnetic wave having a specific frequency, and to adapt the frequency of the electrical signal coming from the independent radiofrequency receiver coil to a frequency of the imager.

Various other characteristics appear from the following description given with reference to the accompanying drawings which show embodiments of the invention as non-limiting examples.

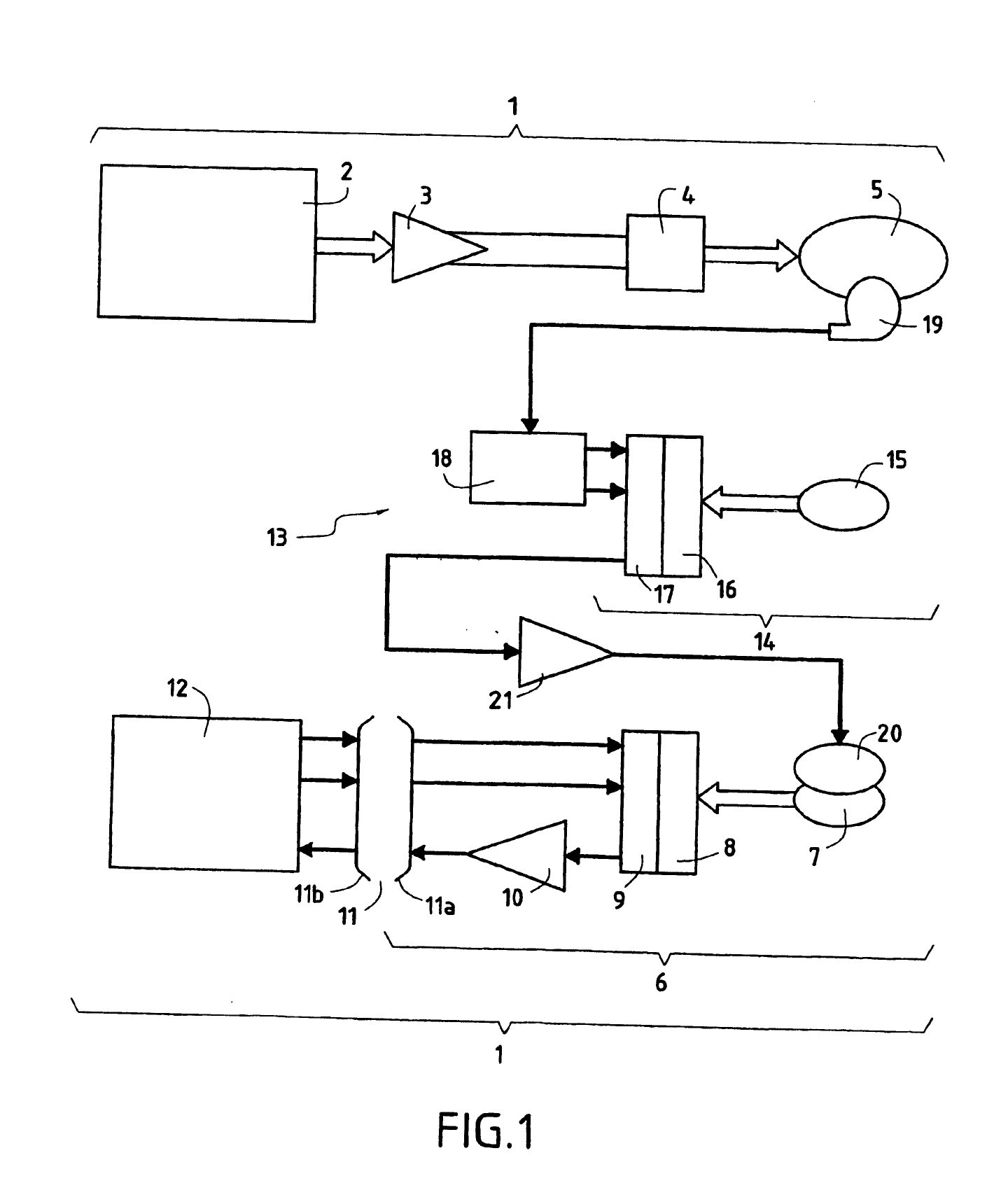

FIG. 1 shows an embodiment of the invention enabling different types of independent radiofrequency receiver coil to be fitted to a given imager.

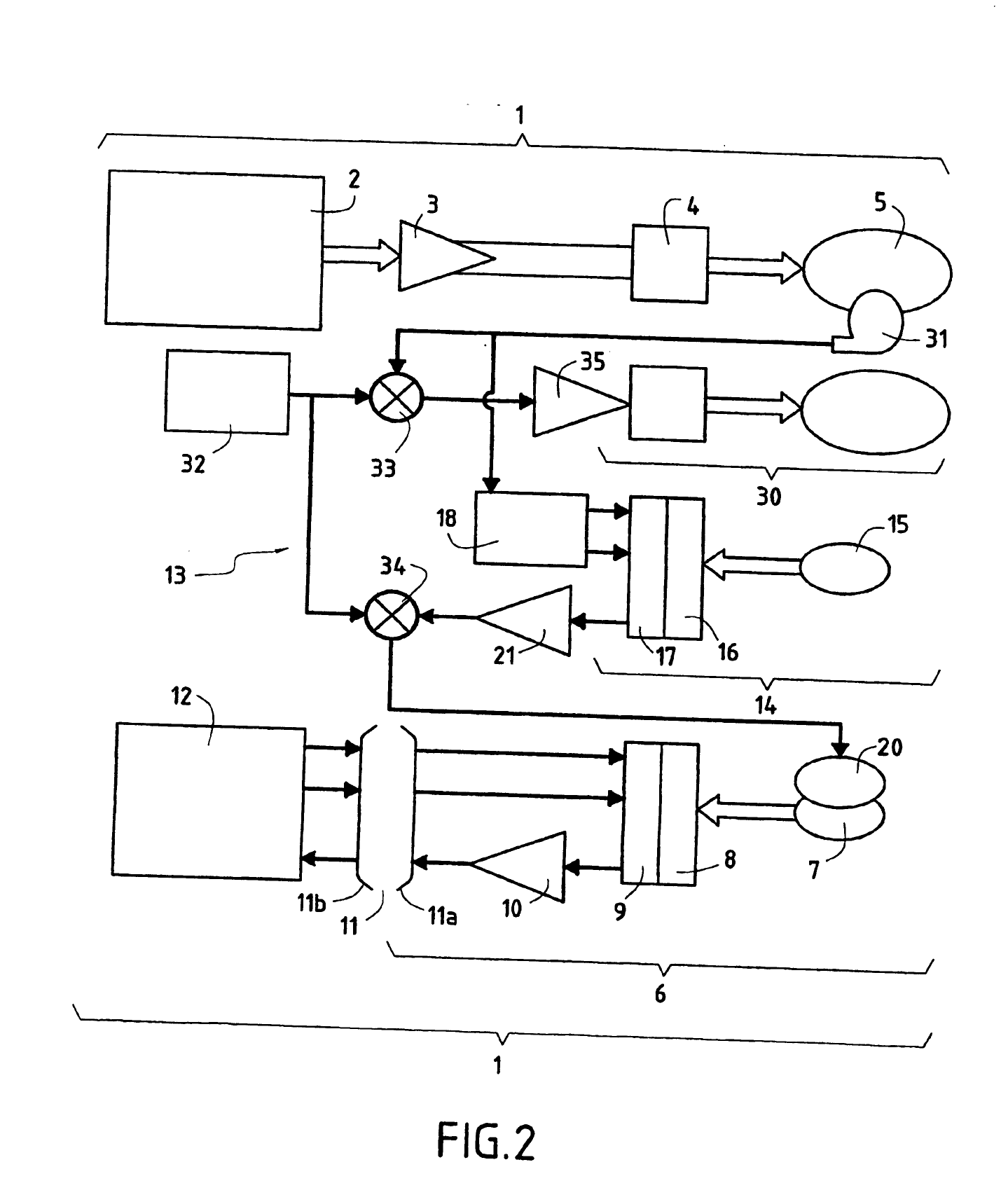

FIG. 2 shows another embodiment of the invention further making it possible to transmit an electromagnetic wave having a specific frequency and to adapt the frequency of the electrical signal delivered by the independent radiofrequency receiver coil to a frequency of the imager.

The embodiment shown in FIG. 1 shows all of the components of an imager given overall reference 1. One of the functions of the imager 1 is to transmit an electromagnetic wave of given amplitude and frequency. This function is provided by a transmitter system that generally forms an integral portion of the imager 1. At its upstream end the transmitter system comprises a control system 2 for generating an electrical signal which is subsequently amplified by an amplifier 3 connected to said control system 2. Tuning and matching means 4 are disposed downstream from the amplifier in order to filter the amplified signal and optimize the efficiency of amplification. The end of the transmitter system is connected to a radiofrequency transmitter coil 5 for transforming the amplified, matched, and tuned electrical signal into an electromagnetic wave. The radiofrequency transmitter coil 5 is generally inserted in the magnetic field generated by a magnet (not shown). In most cases, the transmitter coil 5 is disposed in such a manner as to surround the item under investigation within said magnetic field. The characteristics of the electromagnetic wave transmitted by the radiofrequency transmitter coil 5 are generally a frequency substantially equal to 64 megahertz (MHz) with a passband of approximately 200 kilohertz (kHz) for a magnetic field of 1.5 teslas (T). Nevertheless, these characteristics may vary as a function of the magnetic field used, and also as a function of the type of analysis performed.

The imager 1 may include means enabling different types of radiofrequency coil to be fitted thereto. In addition, it may include other means enabling adjustment parameters of the tuning and matching means 4 and also of the amplifier 3 to be varied.

As shown in FIG. 1, the imager 1 includes at least one standard radiofrequency receiver coil 6. The standard radiofrequency receiver coil 6 may itself comprise a coil proper 7, tuning and matching means 8 and decoupling means 9. It may also include a preamplifier 10.

In general, the standard radiofrequency receiver coil 6 forms part of a set of standard radiofrequency receiver coils each of which is provided with a standard connector 11a suitable for fitting to the connector 11b of the model of imager in use. The connection system 11 of the standard radiofrequency receiver coils associated with a particular model of imager can vary as a function of the model and of the design date of said coil and/or of the imager in use.

The imager 1 also includes a radiofrequency receiver 12 provided with the connector 11b enabling the electrical signal coming from the standard radiofrequency receiver coil to be received.

Naturally, the imager 1 includes digital processor means (not shown) for building two- or three-dimensional images of the item under investigation.

In accordance with the invention, the nuclear magnetic resonance imager is associated with a universal connector/receiver device 13 comprising at least one independent radiofrequency receiver coil 14. This coil is independent in that it does not constitute a component of the imager 1 as described above. The independent radiofrequency receiver coil 14 is fitted with a coil proper 15 for transforming said signal induced by resonance into an electrical signal. This coil 14, and in particular the coil proper 15, is adapted to the shape and/or size of the item under investigation. The independent radiofrequency receiver coil can be positioned so as to surround the item under investigation. It may also be placed close to the item under investigation. Generally, like the item under investigation, it is disposed within the magnetic field generated by the magnet associated with the imager. In addition, it may advantageously be disposed inside the radiofrequency transmitter coil 5.

The independent radiofrequency receiver coil 14 preferably comprises tuning and matching means 16 to which the coil proper 15 is attached. In particular, said independent radiofrequency receiver coil 14 may include decoupling means 17. These decoupling means 17, also referred to as isolating devices, serve to minimize coupling between the radiofrequency transmitter coil 5 and the independent radiofrequency receiver coil 14. Such coupling has two harmful consequences:
the first is to reinforce the field induced by the transmitted electromagnetic wave, the independent radiofrequency receiver coil 14 then acting as a magnetic flux concentrator which disturbs the normal operation of the imager; and
the second is caused by unacceptable heating of the item under investigation which, in the medical field, generally corresponds to a portion of the body of a patient, where heating can endanger the patient if a certain limit is exceeded.

In an advantageous embodiment, an electronic control circuit 18 is connected to the tuning and matching means 16 and/or to the decoupling means 17. The electronic control circuit 18 serves in particular to adjust the tuning and matching means 16 and the decoupling means 17.

The device preferably comprises a monitor loop 19 providing magnetic coupling with the radiofrequency transmitter coil 5 and connected to the electronic control circuit 18. The monitor loop 19 serves in particular to servo-control the decoupling means 17 of the independent radiofrequency receiver coil 14 relative to the electromagnetic wave transmitted by the radiofrequency transmitter coil 5, and to do so by means of the electronic circuit 18.

The magnetic coupling of the monitor loop 19 with the radiofrequency transmitter coil 5 can be implemented in any way. This coupling makes it possible to transfer, in the form of an electrical signal, the electromagnetic wave transmitted by the radiofrequency transmitter coil 5 to the electronic control circuit 18.

In a particular embodiment, the independent radiofrequency receiver coil 14 is selected from standard radiofrequency receiver coils that are available on the market.

In accordance with the invention, the independent radiofrequency receiver coil 14 is connected to a receiver coupling loop 20 providing magnetic coupling with the standard radiofrequency receiver coil 6.

It follows that for a given imager, it is possible to use any type of independent radiofrequency receiver coil 14, regardless of the model and the design date of the imager.

The receiver coupling loop 20 can be positioned close to or preferably inside the standard radiofrequency receiver coil 7 proper.

Generally, the receiver coupling loop 20 and the standard radiofrequency receiver coil 6 are both located outside the magnetic field generated by the magnet associated with the imager. Under such circumstances, the function of the receiver coupling loop 20 is to transfer an electrical signal coming from the independent radiofrequency receiver coil 14.

In another method of use, the receiver coupling loop 20 and the standard radiofrequency receiver coil 6 are located within the magnetic field generated by the magnet associated with the imager. The receiver coupling loop 20 and the coil 7 then serve not only to transfer an electrical signal coming from the independent radiofrequency receiver coil 14, but also to receive the electromagnetic signal induced by nuclear magnetic resonance in the item under investigation.

A coupling preamplifier 21 is preferably inserted between the independent radiofrequency receiver coil 14 and the receiver coupling loop 20.

It is possible to envisage a device having a plurality of independent radiofrequency receiver coils 14 connected by magnetic coupling via as many receiver coupling loops 20 with as many standard radiofrequency receiver coils 16 themselves connected to the imager.

In a particular embodiment of the invention, two independent radiofrequency receiver coils 14 disposed in quadrature are connected to two receiver coupling loops 20 providing magnetic coupling with two standard radiofrequency receiver coils 6 of the imager 1. By placing the independent radiofrequency receiver coils 14 in quadrature, each of them is disposed in such a manner that the main axes of the fields form an angle of approximately 90°. In this particular configuration, it is generally necessary to provide a device that enables the electrical signals coming from the standard radiofrequency receiver coils 6 to be added together.

In a variant of the preceding embodiment, two independent radiofrequency receiver coils 14 placed in quadrature are connected to the inputs of an adder device (not shown) having at least two inputs and one output, said output being connected to the receiver coupling loop 20. This embodiment enables a single receiver coupling loop 20 and a single standard radiofrequency receiver coil 6 to be used. It should be observed that it is possible to envisage using a number of independent radiofrequency receiver coils 14 that is greater than two.

In a particular embodiment of the invention, the universal connector/receiver device enables an electromagnetic wave to be transmitted at a specific frequency different from the frequency of the imager, and to adapt the frequency of the electrical signal coming from the independent radiofrequency receiver coil 14 to the frequency of the imager. This also makes it possible to cause nuclei other than those of hydrogen atoms to enter into resonance, where the resonant frequency of hydrogen is approximately 42 MHz, for a magnetic field of 1 T. As a result, it is possible to envisage causing the nuclei of molecules such as phosphorus or helium to enter into resonance. In addition, progress in the field of nuclear magnetic resonance imaging is tending towards using ever stronger magnetic fields, e.g. 3 T fields, such that the frequencies of the transmitted electromagnetic waves need to be adapted accordingly.

As shown in FIG. 2, in this particular embodiment of the invention, the universal connector/receiver device 13 includes at least one independent radiofrequency transmitter coil 30 connected to at least one transmitter coupling loop 31 providing magnetic coupling with the radiofrequency transmitter coil 5. The universal receiver device 13 further comprises a frequency synthesizer 32 interposed between the output from the transmitter coupling loop 31 and the input of the receiver coupling loop 20. The frequency synthesizer 32 enables an electromagnetic wave having a specific frequency to be transmitted and enables the frequency of the electrical signal coming from the independent radiofrequency receiver coil 14 to be adapted to a frequency of the imager 1. The frequency synthesizer 32 is interposed at the output of the transmitter coupling loop 31 by means of a frequency adapter device 33 enabling the frequency of the electrical signal which depends on the imager 1 into a specific frequency. Similarly, the synthesizer 32 is interposed at the input of the receiver coupling loop 20 by means of another frequency adapter device 34 (e.g. a mixer) enabling the frequency of the electrical signal coming from the independent radiofrequency receiver coil 14 to be adapted to the frequency of the imager 1.

In a preferred embodiment, the frequency adapter device 33 is connected to an independent radiofrequency transmitter coil 30 via an amplifier 35.

In another preferred embodiment, the independent radiofrequency transmitter coil 30 includes tuning and matching means.

The invention makes it possible, advantageously, to use electromagnetic waves of frequency that can be adapted to a specific frequency which is different from the frequency of the imager. This makes it possible to cause nuclei of atoms other than hydrogen, e.g. atoms of phosphorus or helium to be caused to enter into resonance. This also makes it possible to adapt the frequency of the electromagnetic waves to magnetic fields that are stronger than those commonly in use.

The invention is not limited to the examples described and shown since various modifications can be applied thereto without going beyond the ambit of the invention.

1. A universal connector/receiver device for a nuclear magnetic resonance imager (1) provided with at least one radiofrequency transmitter coil (5) and with at least one standard radiofrequency receiver coil (6),
the device being characterized in that it comprises at least one independent radiofrequency receiver coil (14) adapted to the shape and/or size of an item to be investigated, for the purpose of transforming an electromagnetic signal induced by nuclear magnetic resonance into an electrical signal, said independent radiofrequency receiver coil (14) being connected to a receiver coupling loop (20) serving to provide magnetic coupling with the standard radiofrequency receiver coil (6).

2. A device according to claim 1, characterized in that a coupling preamplifier (21) is inserted between the independent radiofrequency receiver coil (14) and the receiver coupling loop (20).

3. A device according to claim 1, characterized in that the independent radiofrequency receiver coil (14) includes tuning and matching means (16).

4. A device according to claim 1, characterized in that the independent radiofrequency receiver coil (14) includes decoupling means (17).

5. A device according to claim 1, characterized in that the independent radiofrequency receiver coil (14) is selected from standard radiofrequency receiver coils.

6. A device according to claim 1, characterized in that the receiver coupling loop (20) is positioned inside the standard radiofrequency receiver coil (6).

7. A device according to claim 1, characterized in that the receiver coupling loop (20) and the standard radiofrequency receiver coil (6) are located within a magnetic field generated by a magnet associated with the imager.

8. A device according to claim 2, characterized in that an electronic control circuit (18) is connected to the tuning and matching means (16) and/or to the decoupling means (17).

9. A device according to claim 1, characterized in that two independent radiofrequency receiver coils (14) disposed in quadrature are connected to two receiver coupling loops (20) providing magnetic coupling with two standard radiofrequency receiver coils (6) of the under imager (1).

10. A device according to claim 1, characterized in that two independent radiofrequency receiver coils (14) disposed in quadrature are connected to the inputs of an adder device having at least two inputs and one output, said output being connected to the receiver coupling loop (20).

11. A device according to claim 8, characterized in that it includes a monitor loop (19) providing magnetic coupling with the radiofrequency transmitter coil (2) and connected to the electronic control circuit (18).

12. A device according to claim 1, characterized in that it includes at least one independent radiofrequency transmitter coil (30) connected to at least one transmitter coupling loop (31) providing magnetic coupling with the radiofrequency transmitter coil (5), and in that a frequency synthesizer (32) is interposed between the output of the transmitter coupling loop (31) and an input of the receiver coupling loop (20) in order to transmit an electromagnetic wave having a specific frequency, and to adapt the frequency of the electrical signal coming from the independent radiofrequency receiver coil (14) to a frequency of the imager (1).

13. A device according to claim 12, characterized in that the independent radiofrequency transmitter coil (30) is fed via an amplifier (35) which is interposed between the transmitter coupling loop (31) and said independent radiofrequency transmitter coil (30).

14. A device according to claim 12, characterized in that the independent radiofrequency transmitter coil (30) includes tuning and matching means.
